(19) United States
(12) Patent Application Publication
(10) Pub. No.: US 2022/0031172 A1
(43) Pub. Date: Feb. 3, 2022

(54) Smart Device-Based Radar System Detecting Human Vital Signs Using a Compact Circularly-Polarized Antenna

(71) Applicant: Google LLC, Mountain View, CA (US)

(72) Inventors: Yuchu He, Mountain View, CA (US); Jiang Zhu, Cupertino, CA (US); Changzhan Gu, Milpitas, CA (US); Haijiang Ma, Campbell, CA (US)

(73) Assignee: Google LLC, Mountain View, CA (US)

(21) Appl. No.: 17/299,271

(22) PCT Filed: Dec. 24, 2018
(86) PCT No.: PCT/US2018/067455

§ 371 Date: Jun. 2, 2021

Related U.S. Application Data
(60) Provisional application No. 62/775,606, filed on Dec. 5, 2018.

(51) Int. Cl. A61B 5/0205; G01S 7/02; G01S 7/35

(52) U.S. Cl. CPC A61B 5/0205; G01S 7/026; A61B 5/6802; G01S 7/352; G01S 7/028

(57) ABSTRACT

Techniques and apparatuses are described that implement a smart device-based radar system capable of detecting human vital signs using a compact circularly-polarized antenna. A radar system includes at least one compact circularly-polarized antenna with a patch antenna and a quadrature hybrid coupler. The quadrature hybrid coupler is on a separate plane that is above or below the patch antenna. By vertically stacking the patch antenna with the quadrature hybrid coupler, lateral dimensions of the radar system can be reduced relative to other antenna configurations or designs. The quadrature hybrid coupler can also have a ring-shape design to improve isolation and polarization purity of the compact circularly-polarized antenna relative to a rectangular design. The compact circularly-polarized antenna enables a radar system operating at frequencies between approximately 1 gigahertz (GHz) and 24 GHz to be implemented within a variety of small smart devices.

### CROSS-REFERENCE TO RELATED APPLICATION

This application claims the benefit of U.S. Provisional Application No. 62/775,606 filed 5 Dec. 2018, the disclosure of which is hereby incorporated by reference in its entirety herein.

### BACKGROUND

Radars are useful devices that can detect and track objects. While radar is a common tool used in military and air-traffic-control operations, technological advances are making it possible to integrate radars in small commercial devices. In many cases, a radar may replace bulky and expensive sensors, such as a camera, and provide improved performance in the presence of different environmental conditions, such as low lighting and fog, or with moving or overlapping targets. While it may be advantageous to use radar, there are many challenges associated with integrating radar in commercial devices.

One such challenge involves restrictions that a small consumer device may impose on a radar's design. To satisfy size or layout constraints using linearly-polarized antennas, for example, smaller antenna element spacings may be used. However, smaller antenna element spacings can increase mutual coupling between the antenna elements and result in degraded signal-to-noise ratio performance, which may make it challenging to achieve sufficient accuracies or detect weak reflections for some applications. As such, effective operation and capability of a radar integrated within a consumer device may be significantly reduced, which may limit the types of applications the radar can support or the types of consumer devices in which the radar can be implemented.

### SUMMARY

Techniques and apparatuses are described that implement a smart device-based radar system capable of detecting human vital signs using a compact circularly-polarized antenna. A radar system includes at least one compact circularly-polarized antenna with a patch antenna and a quadrature hybrid coupler. The quadrature hybrid coupler is on a separate plane that is above or below the patch antenna. By vertically stacking the patch antenna with the quadrature hybrid coupler, lateral dimensions of the radar system can be reduced relative to other designs that have separate transmitting and receiving antennas, high-gain linear phased arrays, or a circularly-polarized antenna with an edge-feed configuration. The quadrature hybrid coupler can have a ring-shape design and be implemented using microstrip transmission lines or a surface-mount device. The ring-shape design of the quadrature hybrid coupler improves isolation and polarization purity of the compact circularly-polarized antenna relative to a rectangular design. The resulting compact circularly-polarized antenna can have relatively small lateral dimensions for frequencies between approximately 1 gigahertz (GHz) and 24 GHz, for instance, thereby enabling the radar system to be implemented within a variety of space-constrained smart devices.

Aspects described below include a smart device comprising a radar system and a human vital-sign detection module. The radar system includes at least one compact circularly-polarized antenna, a transmitter, and a receiver. The compact circularly-polarized antenna includes a patch antenna positioned on a first plane and having a first feed pin and a second feed pin. The compact circularly-polarized antenna also includes a quadrature hybrid coupler positioned on a second plane such that at least a portion of the quadrature hybrid coupler on the second plane overlaps a portion of the patch antenna on the first plane along an axis that is perpendicular to the first and second planes. The quadrature hybrid coupler includes a first port, a second port coupled to the first feed pin of the patch antenna, a third port coupled to the second feed pin of the patch antenna, and a fourth port. The transmitter is coupled to the first port of the quadrature hybrid coupler and is configured to transmit, via the at least one compact circularly-polarized antenna, a radar transmit signal with a first circular polarization. The receiver is coupled to the fourth port of the quadrature hybrid coupler and is configured to receive, via the at least one compact circularly-polarized antenna, a radar receive signal with a second circular polarization that is orthogonal to the first circular polarization. The radar receive signal includes a portion of the radar transmit signal that is reflected by a user. The human vital-sign detection module is configured to detect a vital sign of the user based on the radar receive signal.

Aspects described below also include a compact circularly-polarized antenna with a transmit port, a receive port, a patch antenna, and a quadrature hybrid coupler. The transmit port is configured to accept a radar transmit signal from a transmitter and the receive port is configured to provide a radar receive signal to a receiver. The quadrature hybrid coupler is coupled between the patch antenna, the transmit port, and the receive port. The quadrature hybrid coupler and the patch antenna are vertically stacked together and are jointly configured to transmit the radar transmit signal with a first circular polarization and receive the radar receive signal with a second circular polarization that is orthogonal to the first circular polarization.

Aspects described below also include a method that detects human vial signs using a compact circularly-polarized antenna. The method includes transmitting a radar transmit signal with a first circular polarization using a compact circularly-polarized antenna. The compact circularly-polarized antenna includes a patch antenna and a quadrature hybrid coupler, which are vertically stacked together. The method also includes receiving a radar receive signal with a second circular polarization using the compact circularly-polarized antenna. The second circular polarization is orthogonal to the first circular polarization and the radar receive signal includes a portion of the radar transmit signal that is reflected by a user. The method further includes detecting a vital sign of the user based on the radar receive signal.

Aspects described below also include a system with compact means for transmitting and receiving radar signals of orthogonal polarizations.

### BRIEF DESCRIPTION OF THE DRAWINGS

Apparatuses for and techniques implementing a smart device-based radar system capable of detecting human vital signs using a compact circularly-polarized antenna are described with reference to the following drawings. The same numbers are used throughout the drawings to reference like features and components:

FIG. 1 illustrates example environments in which a smart device-based radar system capable of detecting human vital signs using a compact circularly-polarized antenna can be implemented.

FIG. 2 illustrates an example radar system as part of a smart device.

FIG. 3 illustrates an example compact circularly-polarized antenna as part of a radar system.

FIG. 4 illustrates an example ring-shaped quadrature hybrid coupler.

FIG. 5-1 illustrates an example compact circularly-polarized antenna with a microstrip transmission line-based quadrature hybrid coupler.

FIG. 5-2 illustrates another example compact circularly-polarized antenna with a surface-mount quadrature hybrid coupler.

FIG. 6 illustrates an example method for performing operations of a smart device-based radar system capable of detecting human vital signs using a compact circularly-polarized antenna.

FIG. 7 illustrates an example computing system embodying, or in which techniques may be implemented that enable use of, a radar system capable of detecting human vital signs using a compact circularly-polarized antenna.

### DETAILED DESCRIPTION

Overview

Radar systems can be applied to various emerging applications, such as an automotive collision avoidance radar, a healthcare sensor, or motion detectors. A radar can also be used for home health monitoring and provide fall detection, vital-sign detection, or sleep monitoring. One type of radar is a continuous-wave Doppler radar that uses separate transmitting and receiving antennas. In some examples, the transmitting and receiving antennas are linearly-polarized antennas. These antennas, however, can be vulnerable to interference due to mutual coupling between the transmitting and receiving antennas. For instance, a strong signal from the transmitting antenna can leak into the receiving antenna and dominate a reflected signal from an object if the antennas are close to each other and are configured for a same polarization. To improve isolation, cross-polarized linear antennas can be used. However, if a polarization of a reflected signal does not match a polarization of the receiving antenna, it can be challenging for the radar system to detect weak reflected signals. Alternatively, co-polarized transmitting and receiving antennas can be physically separated by a sufficient distance to achieve a desired sensitivity level. However, this can increase a footprint of the radar system and make it impractical for the radar system to fit within some compact devices, such as a smart device.

One technique shrinks an overall size of the radar system by using high frequencies that are greater than or equal to approximately 24 gigahertz (GHz) (e.g., frequencies associated with millimeter wavelengths). Since an antenna that is tuned for these high frequencies is generally smaller in size relative to another antenna that is tuned for low frequencies (e.g., frequencies below 24 GHz), the overall footprint of the radar system is reduced. However, there are several disadvantages to operating the radar system at higher frequencies. The fabrication tolerance can be tighter and the cost of fabrication and circuit components can be higher, for instance. Additionally, high-frequency circuits can be less power efficient and high-frequency signals can experience a larger amount of attenuation due to free space path loss.

To achieve a desired sensitivity at high frequencies and compensate for the free space path loss, the transmitting and receiving antennas can be implemented with high-gain phased arrays consisting of multiple antenna elements. The high-gain phased arrays increase respective gains of the transmitting and receiving antennas to compensate for the additional free space path loss at high frequencies. With the high-gain phased arrays, the radar system can also use beamsteering to detect objects across a wide angular range or focus radiation energy in a particular direction. A resulting size of a high-frequency radar system can be large due to the multiple antenna elements within the high-gain phase arrays. Thus, the advantage of having smaller sized antennas or antenna elements at high frequencies may not result in a radar system having a small footprint if the transmitting and receiving antennas are implemented with high-gain phased arrays.

There are also other challenges involved with designing a radar that operates at high frequencies. In particular, there are target guidelines determined by the Federal Communications Commission (FCC) that specify specific absorption rate (SAR) limits and maximum permitted exposure (MPE) limits Because the specific absorption rate generally increases as frequency increases, a transmit power level or a duration for which the high-frequency radar system operates may be constrained to meet targeted SAR guidelines. Consequently, an overall effectiveness and performance of the high-frequency radar system is reduced. The use of high-gain phased arrays or beamsteering also increases power densities, which can make it challenging for the high-frequency radar system to meet targeted MPE limits Even if the high-frequency radar system meets the FCC's guidelines, users may choose to not purchase a smart device with the high-frequency radar system to mitigate their exposure to high-frequency radiation. Therefore, it may not be desirable to design a radar system that operates at high frequencies to decrease a size of the radar system.

Another technique implements the transmitting antenna and the receiving antenna with a single circularly-polarized antenna. The circularly-polarized antenna reduces the overall footprint of the radar system by reducing a quantity of antennas. To create the circularly-polarized antenna, some techniques connect a patch antenna and a quadrature hybrid coupler together using an edge-feed configuration. As such, the quadrature hybrid coupler is placed in a same plane as the edge-fed patch antenna. Although this design may be simple, the quadrature hybrid coupler can have a significant footprint and hence, the overall lateral dimension of the radar system can be large. This may work for high-frequency radar systems that operate at frequencies above 24 GHz as sizes of the edge-fed patch antenna and the quadrature hybrid coupler can be small. However, this type of configuration can result in a relatively large footprint for low-frequency radar systems, such as those that operate at frequencies below 24 GHz.

In contrast, techniques described herein present a smart device-based radar system capable of detecting human vital signs using a compact circularly-polarized antenna. A radar system includes at least one compact circularly-polarized antenna with a patch antenna and a quadrature hybrid coupler. The quadrature hybrid coupler is on a separate plane that is above or below the patch antenna. By vertically stacking the patch antenna with the quadrature hybrid coupler, lateral dimensions of the radar system can be reduced relative to other designs that have separate transmitting and receiving antennas, high-gain linear phased arrays, or a circularly-polarized antenna with an edge-feed configuration. The quadrature hybrid coupler can have a ring-shape design and be implemented using microstrip transmission lines or a surface-mount device. The ring-shape design of the quadrature hybrid coupler improves isolation and polarization purity of the compact circularly-polarized antenna relative to a rectangular design. The resulting compact circularly-polarized antenna can have relatively small lateral dimensions for frequencies between approximately 1 gigahertz (GHz) and 24 GHz, for instance, thereby enabling the radar system to be implemented within a variety of space-constrained smart devices. As an example, the compact circularly-polarized antenna has dimensions of approximately 50 millimeters (mm) by 50 mm by 3 mm for frequencies at approximately 2.4 GHz.

Example Environment

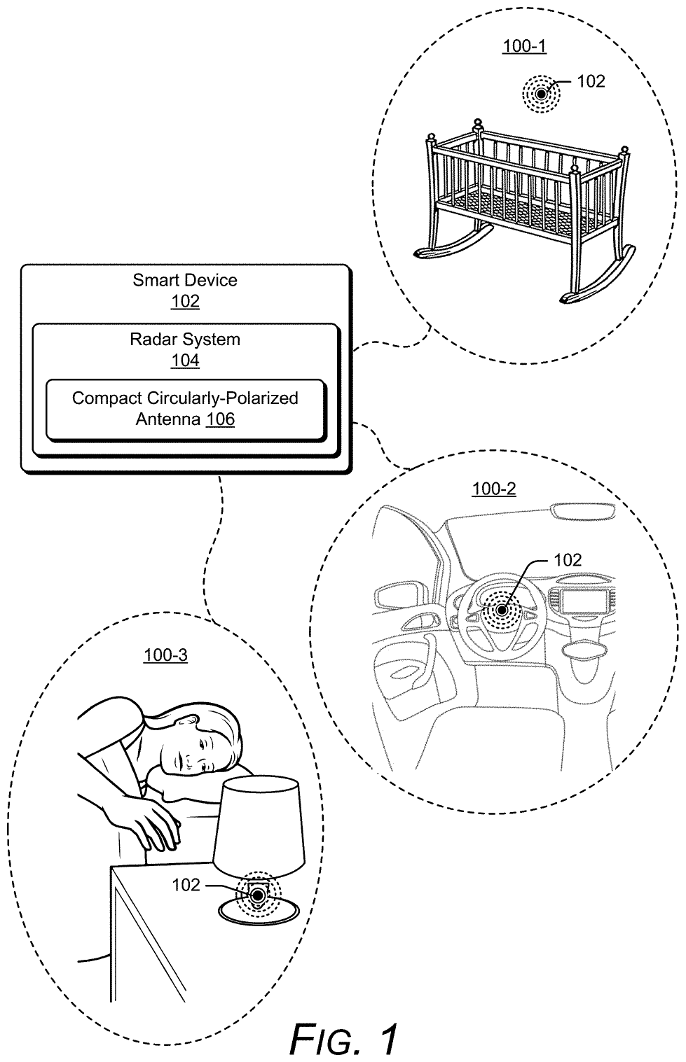

FIG. 1 is an illustration of example environments in which techniques using, and an apparatus including, a smart device-based radar system capable of detecting human vital signs using a compact circularly-polarized antenna may be embodied. In the depicted environments 100-1, 100-2, and 100-3, a smart device 102 includes a radar system 104 with at least one compact circularly-polarized antenna 106. The compact circularly-polarized antenna 106 enables a size of the radar system 104 to fit within size constraints of the smart device 102.

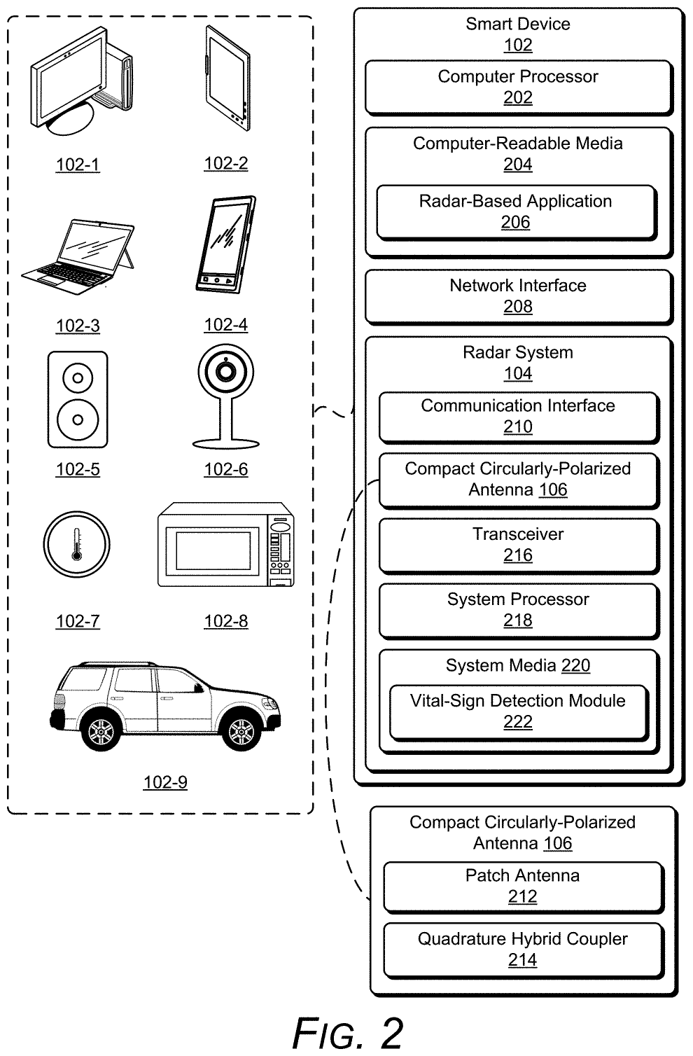

Some implementations of the radar system 104 are particularly advantageous as applied in the context of human vital-sign health monitoring systems, for which there is a convergence of issues such as a need for low power, limitations in a spacing and layout of the radar system 104, and other issues. Although the implementations are particularly advantageous in the described context of a system for which human vital-sign monitoring is required, it is to be appreciated that the applicability of the features and advantages of the present invention is not necessarily so limited, and other implementations involving other types of electronic devices may also be within the scope of the present teachings. Although the smart device 102 is shown as different household or vehicle objects in FIG. 1, the smart device 102 can be implemented as any suitable computing or electronic device, as described in further detail with respect to FIG. 2.

Exemplary power consumption of the radar system 104 may be on the order of a few milliwatts to several milliwatts (e.g., between approximately two milliwatts and twenty milliwatts). The requirement of such a limited footprint for the radar system 104, which is needed to accommodate the many other desirable features of the smart device 102 in such a space-limited package (e.g., a camera sensor, a fingerprint sensor, a display, and so forth) combined with power limitations, can lead to compromises in the accuracy, power efficiency, and efficacy of human vital-sign detection, at least some of which can be overcome in view of the teachings herein.

In the environment 100-1, the smart device 102 is a baby monitor that is mounted to a wall. Using the radar system 104, the smart device 102 monitors a baby's vital signs while the baby is in a crib. In particular, the radar system 104 determines the baby's heart rate and respiration rate. The smart device 102 notifies a parent if the radar system 104 detects an abnormality or change in the baby's vital signs. The smart device 102 can send a wireless communication message to the parent's smartphone or produce an audible sound, for instance. In this manner, the smart device 102 alerts the parent if the baby is having difficulty breathing, if the baby wakes up from a nap, or if the baby escaped from the crib.

In the environment 100-2, the smart device 102 is a steering wheel of a vehicle. Using the radar system 104, the smart device 102 monitors a driver's vital signs. If the radar system 104 determines that the driver's heartbeat or respiration rate is indicative of the driver falling asleep, the smart device 102 produces an audible tone to wake up the driver. If the driver has a heart attack while driving or becomes unconscious due to a vehicle accident, the radar system 104 detects the driver's condition and the smart device 102 proactively notifies emergency services.

In the environment 100-3, the smart device 102 is a lamp that monitors a user's sleep cycle. Based on the human vital signs measured by the radar system 104, the smart device 102 can determine an appropriate time to wake the user during the user's sleep cycle. The smart device 102 can also maintain a history of the user's sleep habits for future analysis.

Further to the descriptions above, a user may be provided with controls allowing the user to make an election as to both if and when systems, programs, or features described herein may enable collection of user information (e.g., information about a user's vital signs, a user's movement, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

In the above example environments, the compact circularly-polarized antenna 106 transmits and receives radar signals having frequencies between approximately 2 and 6 GHz to detect human vital signs. Isolation performance of the compact circularly-polarized antenna 106 enables a human's heartbeat and respiration rate to be detected at ranges varying from 15 centimeters (cm) to 60 cm with a transmit power of approximately 0 decibel-milliwatts (dBm) and with a relatively constant transmit frequency of 2.4 GHz. The use of low frequencies (e.g., frequencies below approximately 24 GHz) is also desirable to reduce the specific absorption rate experienced by the user relative to high frequencies (e.g., frequencies at or above approximately 24 GHz or extremely-high frequencies with millimeter wavelengths) and to readily meet targeted guidelines, such as those determined by the FCC, while achieving desired performance for human vital-sign detection.

In some situations, the radar system 104 also uses these lower frequencies to detect a presence of one or more users, track one or more users around the smart device 102, or recognize a user's gesture for touch-free control. For these types of applications, the radar system 104 uses the compact circularly-polarized antenna to transmit a different type of signal, such as a linear-frequency modulated (e.g., chirp) signal, which enables the radar system 102 to determine both position and movement information of the user. Based on radar data collected by the radar system 104, the smart device 102 performs one or more actions (e.g., displays new content, moves a cursor, activates one or more sensors, or opens an application). In other situations, a design of the compact circularly-polarized antenna 106 is adapted for high frequencies and large bandwidths to support micro-gesture recognition, as further described below.

FIG. 2 illustrates the radar system 104 as part of the smart device 102. The smart device 102 can be any suitable computing device or electronic device, such as a desktop computer 102-1, a tablet 102-2, a laptop 102-3, a smartphone 102-4, a smart speaker 102-5, a security camera 102-6, a smart thermostat 102-7, a microwave 102-8, and a vehicle 102-9. Other devices may also be used, such as home-service devices, baby monitors, Wi-Fi™ routers, computing watches, computing glasses, gaming systems, televisions, drones, track pads, drawing pads, netbooks, e-readers, home-automation and control systems, and other home appliances. The smart device 102 can be wearable, non-wearable but mobile, or relatively immobile (e.g., desktops and appliances). The radar system 104 can be used as a stand-alone radar system or used with, or embedded within, many different computing devices or peripherals, such as in control panels that control home appliances and systems, in automobiles to control internal functions (e.g., volume, cruise control, or even driving of the car), or as an attachment to a laptop computer to control computing applications on the laptop.

The smart device 102 includes one or more computer processors 202 and computer-readable media 204, which includes memory media and storage media. Applications and/or an operating system (not shown) embodied as computer-readable instructions on the computer-readable media 204 can be executed by the computer processor 202 to provide some of the functionalities described herein. The computer-readable media 204 also includes a radar-based application 206, which uses radar data generated by the radar system 104 to perform a function, such as human vital-sign notification, gesture-based control, presence detection, or collision avoidance for autonomous driving.

The smart device 102 also includes a network interface 208 for communicating data over wired, wireless, or optical networks. For example, the network interface 208 communicates data over a local-area-network (LAN), a wireless local-area-network (WLAN), a personal-area-network (PAN), a wire-area-network (WAN), an intranet, the Internet, a peer-to-peer network, a point-to-point network, a mesh network, and the like. The smart device 102 may also include a display (not shown).

The radar system 104 includes a communication interface 210 to transmit the radar data to a remote device, though this need not be used when the radar system 104 is integrated within the smart device 102. In general, the radar data provided by the communication interface 210 is in a format usable by the radar-based application 206.

The radar system 104 also includes at least one compact circularly-polarized antenna 106 and at least one transceiver 216 to transmit and receive radar signals. In some cases, the radar system 104 includes multiple compact circularly-polarized antennas 106 to implement a multiple-input multiple-output (MIMO) radar capable of transmitting and receiving multiple distinct waveforms at a given time (e.g., a different waveform per compact circularly-polarized antenna 106). The use of multiple waveforms can increase a measurement accuracy of the radar system 104. Multiple compact circularly-polarized antennas 106 can also implement a one-dimensional or two-dimensional antenna array to increase a sensitivity of the radar system 104 or to use analog or digital beamsteering techniques.

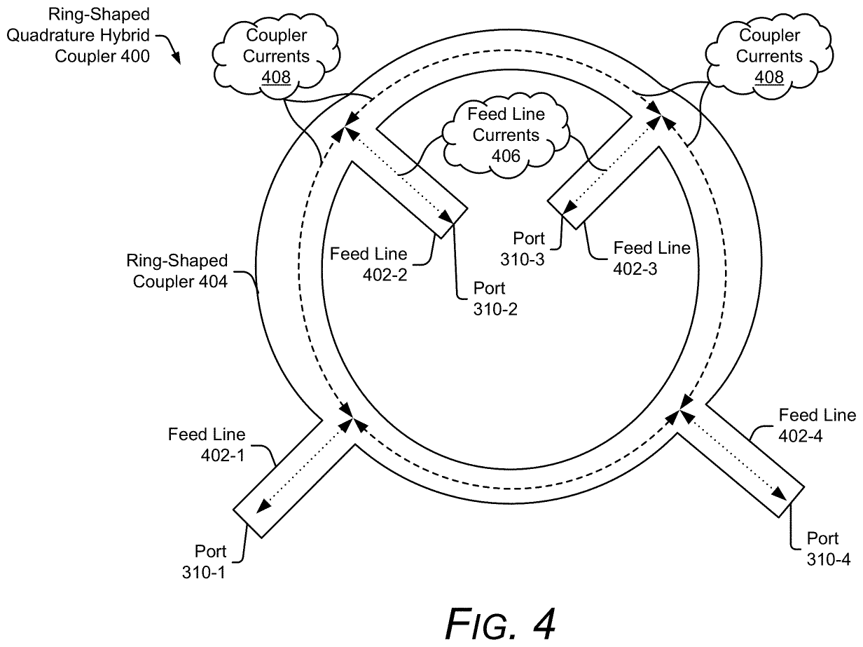

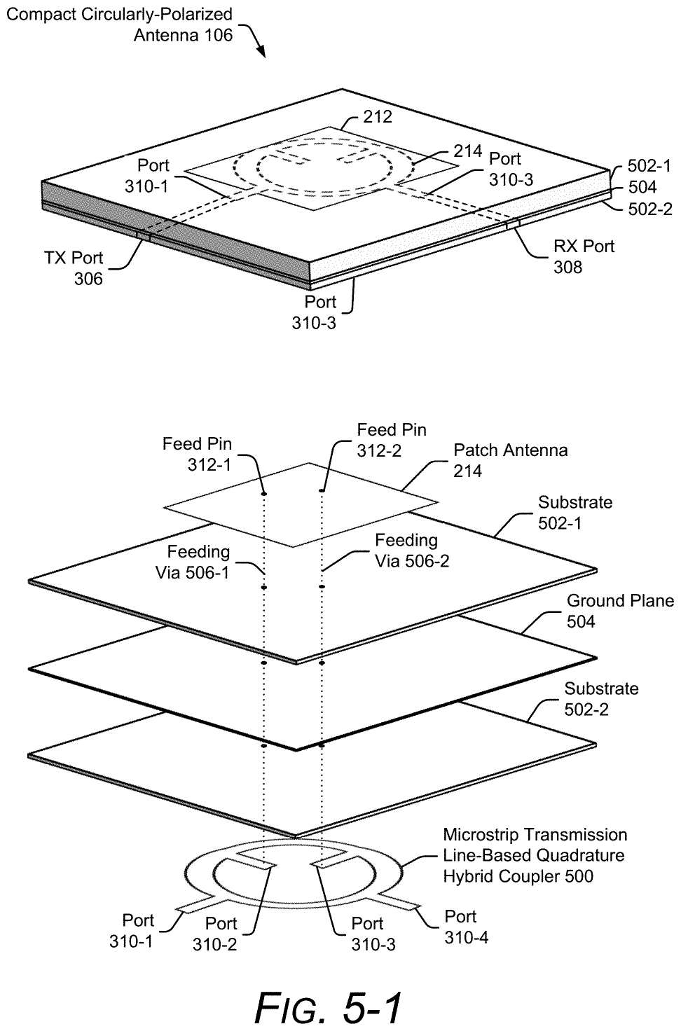

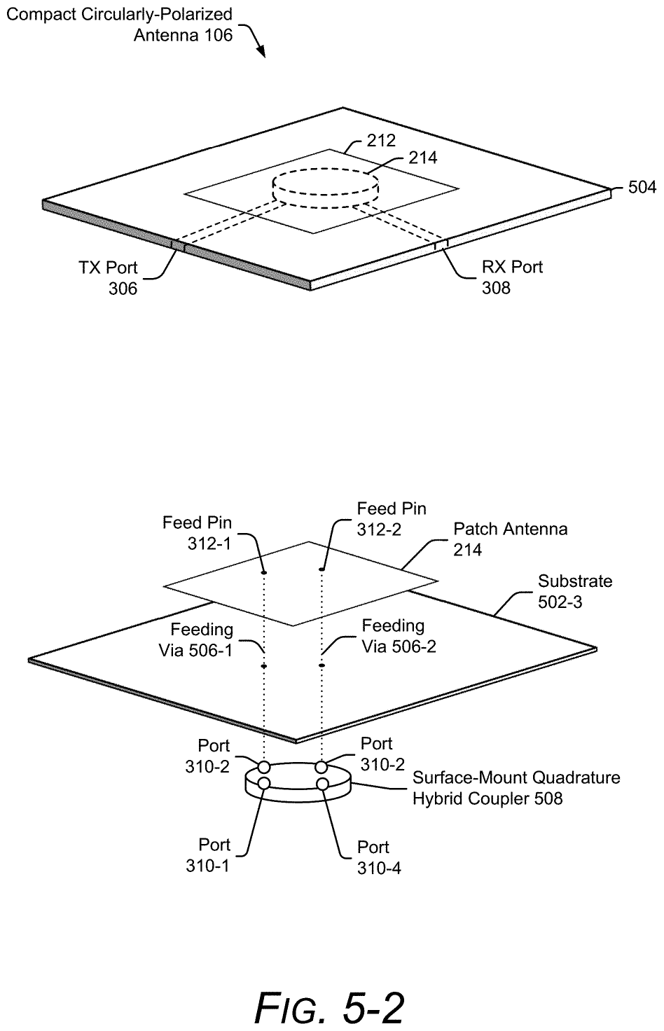

The compact circularly-polarized antenna 106 includes a patch antenna 212 and a quadrature hybrid coupler 214, which are arranged in a vertical fashion. The patch antenna 212 is a dual linearly-polarized patch antenna with orthogonal polarizations. Generally, the patch antenna 212 is a planar antenna that is relatively flat and has either a square (e.g., rectangular) or circular shape. Two ports of the quadrature hybrid coupler 214 have a 90 degree phase difference and are coupled to two feed pins of the patch antenna 212. The two feed pins produce an electric field that varies in two orthogonal dimensions and results in the transmission and reception of circularly-polarized signals of opposite handedness. The quadrature hybrid coupler can be implemented using microstrip transmission lines, as shown in FIG. 5-1, or implemented as a surface-mount device, as shown in FIG. 5-2. In some implementations, the quadrature hybrid coupler 214 is a ring-shaped quadrature hybrid coupler shown in FIG. 4.

The compact circularly-polarized antenna 106 is pin-fed instead of edge-fed so that the quadrature hybrid coupler 214 can be placed on a plane that is above or below the patch antenna 212. In this manner, at least a portion of the quadrature hybrid coupler 214 overlaps a portion of the patch antenna 212 along an axis that is perpendicular to planes of the patch antenna 212 and the quadrature hybrid coupler 214. Generally, the axis is substantially perpendicular to these planes such that an angle between the axis and either one of the planes is between approximately 80 to 100 degrees, 85 to 90 degrees, or 88 to 92 degrees, for instance. This enables the compact circularly-polarized antenna 106 to have a small footprint relative to other circularly-polarized antenna designs that use an edge-feed configuration. With the pin-feed configuration, the feed pins of the patch antenna 212 are connected to the ports of the quadrature hybrid coupler 214 through feeding vias. This enables the quadrature hybrid coupler 214 to be vertically stacked with the patch antenna 212; hence, the lateral footprint of the radar system 104 can be reduced.

The transceiver 216 is indirectly or directly coupled to the one or more compact circularly-polarized antennas 106. The transceiver 216 includes circuitry and logic for transmitting and receiving radar signals via the compact circularly-polarized antenna 106. Components of the transceiver 216 can include amplifiers, mixers, switches, analog-to-digital converters, filters, and so forth for conditioning the radar signals. The transceiver 216 also includes logic to perform in-phase/quadrature (I/Q) operations, such as modulation or demodulation. A variety of modulations can be used to produce the radar signals, including linear frequency modulations, triangular frequency modulations, stepped frequency modulations, or phase modulations. Alternatively, the transceiver can produce radar signals with a relatively constant frequency or a single tone. The transceiver 216 can be configured to support continuous-wave or pulsed radar operations and generate or receive signals with frequencies between approximately 1 and 24 GHz, between approximately 2 and 4 GHz, or at approximately 2.4 GHz. In high-frequency implementations, the transceiver 216 can generate or receive signals with frequencies above 24 GHz.

The radar system 104 may also include one or more system processors 218 and a system media 220 (e.g., one or more computer-readable storage media). Although the system processor 218 is shown to be separate from the transceiver 216 in FIG. 2, the system processor 218 may be implemented within the transceiver 216 as a digital signal processor, for instance. The system processor 218 executes computer-readable instructions that are stored within the system media 220. Example digital operations performed by the system processor 218 include Fast-Fourier Transforms (FFTs), filtering, modulations or demodulations, digital signal generation, and so forth.

The system media 220 includes a vital-sign detection module 222 (e.g., a human vital-sign detection module 222), which determines a heartbeat or a respiration rate of one or more users. As an example, the respiration rate can be at approximately 18 breaths per minute while the heartbeat can be at approximately 65 beats per minute. Since the heartbeat and respiration rate typically have different frequency ranges, the vital sign detection module 222 filters and extracts the different frequencies associated with the heartbeat and respiration rate signals from a received radar signal. The system processor 218 can also perform an FFT to identify frequency peaks that respectively correspond to the respiration rate and the heartbeat. Although not shown, the system media 220 can also include other types of modules, such as a gesture recognition module, a collision avoidance module, a user detection module, and so forth.

Compact Circularly-Polarized Antenna

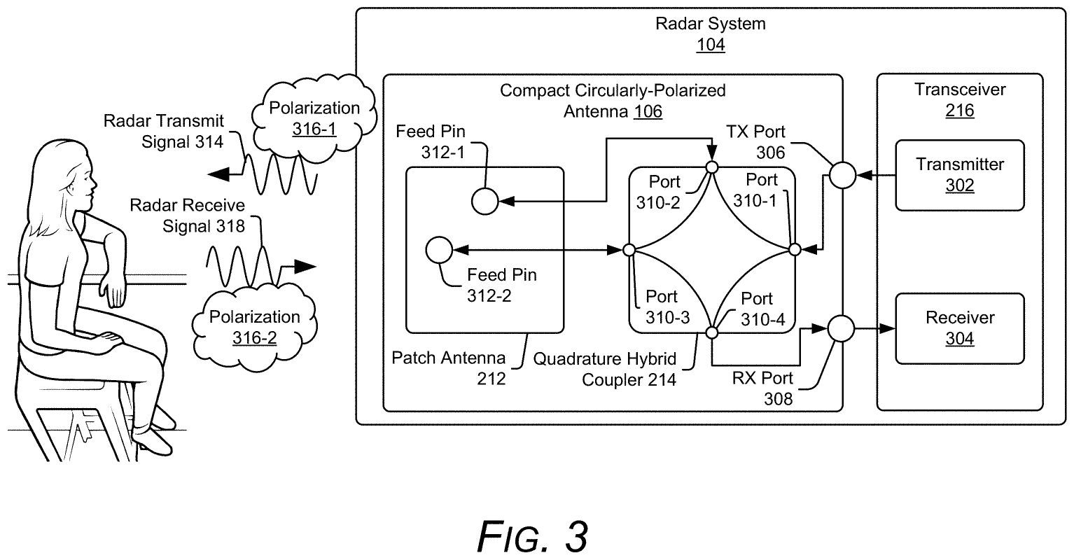

FIG. 3 illustrates the compact circularly-polarized antenna 106 as part of the radar system 104. In the depicted configuration, the transceiver 216 of the radar system 104 includes a transmitter 302 and a receiver 304. The compact circularly-polarized antenna 106 includes a transmit port 306 that is coupled to the transmitter 302 and a receive port 308 that is coupled to the receiver 304. Within the compact circularly-polarized antenna 106, at least a portion of the quadrature hybrid coupler 214 overlaps a portion of the patch antenna 212 along an axis that is relatively perpendicular to planes of the patch antenna 212 and the quadrature hybrid coupler 214. Although the patch antenna 212 and the quadrature hybrid coupler 214 are vertically stacked together, these components are illustrated side-by-side in FIG. 3 for simplicity.

The quadrature hybrid coupler 214 includes four ports 310-1, 310-2, 310-3, and 310-4. The first port 310-1 is coupled to the transmit port 306 and the fourth port 310-4 is coupled to the receive port 308. The second and third ports 310-2 and 310-3 are respectively coupled to feed pins 312-1 and 312-2 of the patch antenna 212. The second and third ports 310-2 and 310-3 have either a +90 degree phase difference or a −90 degree phase difference as seen from the first port 310-1 or the fourth port 310-4. Based on this property, the compact circularly-polarized antenna 106 transmits and receives circularly-polarized signals of opposite handedness (e.g., transmits right-hand circularly-polarized (RHCP) signals and receives left-hand circularly-polarized (LHCP) signals, or transmits LHCP signals and receives RHCP signals).

Isolation between the transmitter 302 and the receiver 304 using the compact circularly-polarized antenna 106 improves relative to many other antenna designs because the compact circularly-polarized antenna 106 transmits and receives orthogonal circular polarizations. This isolation improvement enables the radar system 104 to achieve a desired signal-to-noise ratio (SNR) and a desired sensitivity. Generally, the mutual coupling (e.g., scattering parameter S21) of the compact circularly-polarized antenna 106 can be small relative to other radar systems that use separate linearly-polarized transmitting and receiving antennas. The mutual coupling can be less than approximately −30 decibels (dB), for instance. The compact circularly-polarized antenna 106 also has a symmetric design due to a shape of the patch antenna 212 and the quadrature hybrid coupler 214; hence, exciting either the first port 310-1 or the fourth port 310-4 achieves approximately identical radiation patterns and antenna efficiencies, which are desirable properties in a circularly-polarized antenna design.

For human vital-sign detection, the transmitter 302 generates and provides a continuous-sinusoidal signal at a relatively steady (e.g., approximately constant) frequency to the transmit port 306. From a transmission perspective, the first port 310-1 represents an input port and the fourth port 310-4 represents an isolated port of the quadrature hybrid coupler 214. The second and third ports 310-2 and 310-3 represent either a through port or a coupled port of the quadrature hybrid coupler 214. At the second and third ports 310-2 and 310-3, the quadrature hybrid coupler 214 generates two component signals of the provided signal that have a relatively similar amount of power (e.g., approximately half a power of the provided signal) and have a phase difference of approximately 90 degrees. These signals are respectively provided to the feed pins 312-1 and 312-2 of the patch antenna 212, which causes the compact circularly-polarized antenna 106 to transmit a radar transmit signal 314 with a first circular polarization 316-1. The first circular polarization 316-1 can be a RHCP or a LHCP.

The radar transmit signal 314 impinges on an object (e.g., a user) and a radar receive signal 318 is reflected from the object. Due to a property of circular polarization, a handedness of the incident polarization (e.g., the first circular polarization 316-1) is flipped upon reflection. Consequently, a second circular polarization 316-2 of the radar receive signal 318 is orthogonal to the first circular polarization 316-1. If the first circular polarization 316-1 is a RHCP, then the second circular polarization 316-2 is a LHCP. Due to the Doppler effect, the radar receive signal 318 is a frequency-modulated signal with a frequency that varies according to a respiration rate, heartbeat, and movement of the object.

The compact circularly-polarized antenna 106 receives the radar receive signal 318 via the feed pins 312-1 and 312-2 and provides two component signals of the radar receive signal 318 to the quadrature hybrid coupler 214. From a reception perspective, the first port 310-1 represents an isolated port and the fourth port 310-4 represents an output port of the quadrature hybrid coupler 214. The second and third ports 310-2 and 310-3 represent input ports of the quadrature hybrid coupler 214. The quadrature hybrid coupler 214 appropriately applies a phase offset to these component signals in accordance with the second circular polarization 316-2 and provides a combined signal to the receive port 308. In this way, the compact circularly-polarized antenna 106 receives the radar receive signal 318 with little polarization mismatch (e.g., with a negligible amount of loss). The receiver 304 down-converts the radar receive signal 318 and the radar system 104 determines a relative motion of a human's lungs or heart using digital signal processing.

FIG. 4 illustrates an example ring-shaped quadrature hybrid coupler 400 of the compact circularly-polarized antenna 106. The ring-shaped quadrature hybrid coupler 214 includes multiple feed lines 402-1, 402-2, 402-3, and 402-4, and a ring-shaped coupler 404. The feed lines 402-1 to 402-4 respectively connect the ports 310-1 to 310-4 to the ring-shaped coupler 404. The ring-shaped coupler 404 provides a 90 degree phase shift (e.g., a quarter-wavelength delay) between successive ports 310-1 to 310-4. In other words, a signal excited at port 310-1 has a 90 degree phase offset at port 310-2, a 180 degree phase offset at port 310-3, and a 270 degree phase offset at port 310-4 along a clockwise direction.

Other radar systems may use rectangularly-shaped quadrature hybrid couplers that include a coupler with relatively straight sides and sharp bends at corner junctions that are connected to the feed lines of ports 310-2 and 310-3. These bends, however, can cause impedance mismatch to occur and therefore make it challenging to achieve a desired amount of isolation between ports 310-1 and 310-4. In contrast, the ring-shaped coupler 404 of FIG. 4 is curved throughout, which eliminates the sharp bends at the junctions. This enables feed line currents 406 (shown by the dotted arrows) and coupler currents 408 (shown by the dashed arrows) to be more orthogonal to each other relative to related currents in the rectangular design. As such, coupling between the ring-shaped coupler 404 and the feed lines 402-1 to 402-4 is reduced, which in turn reduces an impedance mismatch of the feed lines 402-1 to 402-4 and improves return loss and isolation.

As an example, an isolation between ports 310-1 and 310-4 of the ring-shaped quadrature hybrid coupler improves by approximately 10 dB relative to the rectangular design. Additionally, both the return losses and the isolation can be below approximately −40 dB at a design frequency (e.g., 2.4 GHz). With these improvements, ports 310-2 and 310-3 of the ring-shaped quadrature hybrid coupler provide a similar amount of power to the patch antenna 212 and have a phase difference of approximately 90 degrees. This further enables the compact circularly-polarized antenna 106 to achieve a desired amount of polarization purity to improve human vital-sign detection performance.

In this design the first and fourth ports 310-1 and 310-4 are located outside a circumference of the ring-shaped coupler 404 while the second and third ports 310-2 and 310-3 are inside the circumference of the ring-shaped coupler 404. This enables the ports 310-2 and 310-3 to be vertically aligned with the feed pins 312-1 and 312-2 of the patch antenna 212 to support the vertical stacking of the quadrature hybrid coupler 214 with the patch antenna 212. Furthermore, the ports 310-1 and 310-4 can be readily coupled to the transmit port 306 and the receive port 308 without the use of vias or traces that overlap or cross over each other. The feed lines 402-1 to 402-4 are also oriented along different radial axes, which cause the feed lines 402-1 to 402-4 to be substantially perpendicular to the ring-shaped coupler 404.

FIG. 5-1 illustrates an example compact circularly-polarized antenna 106 with a microstrip transmission line-based quadrature hybrid coupler 500. In the depicted configuration, the compact circularly-polarized antenna 106 includes the patch antenna 212, a first substrate 502-1, a ground plane 504, a second substrate 502-2, and the microstrip transmission line-based quadrature hybrid coupler 500. In some cases, the microstrip transmission line-based quadrature hybrid coupler 500 is the ring-shaped quadrature hybrid coupler 400 of FIG. 4 implemented with microstrip transmission lines. At the top of FIG. 5-1, the microstrip transmission line-based quadrature hybrid coupler 500 is on a surface of the compact circularly-polarized antenna 106 that is not visible according to the illustrated perspective. At the bottom of FIG. 5-2, an expanded view shows a vertical stacking structure of the compact circularly-polarized antenna 106. For illustration purposes, the expanded view shows the components of the compact circularly-polarized antenna 106 as being physically separated along the vertical dimension. However, this does not necessarily have to be the case, as shown at the top of FIG. 5-1 whereby some surfaces of the components are in contact with each other. In general, planes of the patch antenna 212, the first substrate 502-1, the ground plane 504, the second substrate 502-2, and the microstrip transmission line-based quadrature hybrid coupler 500 are relatively parallel to each other.

In FIG. 5-1, the patch antenna 212 is disposed on a surface of the first substrate 502-1 and the microstrip transmission line-based quadrature hybrid coupler 500 is disposed on a surface of the substrate 502-2. Within the compact circularly-polarized antenna 106, the patch antenna 212, the first substrate 502-1, the ground plane 504, the second substrate 502-2, and microstrip transmission line-based quadrature hybrid coupler 500 are vertically stacked together. Feeding vias 506-1 and 506-2 (represented by dotted lines) connect the ports 310-2 and 310-3 to the feed pins 312-1 and 312-2 through the first substrate 502-1, the ground plane 504, and the second substrate 502-2. With the pin-feed configuration, the ground plane 504 shields the microstrip transmission-line based quadrature hybrid coupler 500 and isolates the feeding structures from the patch antenna 212. This improves the polarization purity of the patch antenna 212 relative to other types of feed configurations.

In one implementation, the substrates 502-1 and 502-2 are implemented using FR4 and have a thickness of approximately 2 mm and 1 mm, respectively. Lateral dimensions of the patch antenna 212 are approximately 28 mm by 28 mm and lateral dimensions of the first substrate 502-1, the ground plane 504, and the second substrate 502-2 are approximately 50 mm by 50 mm. The resulting compact circularly-polarized antenna 106 is therefore 50 mm by 50 mm by 3 mm. This example compact circularly-polarized antenna 106 is designed to transmit and receive signals with frequencies around 2.4 GHz.

FIG. 5-2 illustrates another example compact circularly-polarized antenna 106 with a surface-mount quadrature hybrid coupler 508. In the depicted configuration, the compact circularly-polarized antenna 106 includes the patch antenna 212, a third substrate 502-3, and the surface-mount quadrature hybrid coupler 508. In some cases, the surface-mount quadrature hybrid coupler 508 is the ring-shaped quadrature hybrid coupler 400 of FIG. 4 implemented as a surface-mount device (SMD). At the top of FIG. 5-1, the surface-mount quadrature hybrid coupler 508 is on a surface of the substrate 502-3 that is not visible according to the illustrated perspective. At the bottom of FIG. 5-2, an expanded view shows a vertical stacking structure of the compact circularly-polarized antenna 106. For illustration purposes, the expanded view shows the components of the compact circularly-polarized antenna 106 as being physically separated along the vertical dimension. However, this does not necessarily have to be the case, as shown at the top of FIG. 5-2 whereby some surfaces of the components are in contact with each other. In general, planes of the patch antenna 212, the third substrate 502-3, and the surface-mount quadrature hybrid coupler 508 are relatively parallel to each other.

In FIG. 5-2, the patch antenna 212 is disposed on a first surface of the third substrate 502-3 and the surface-mount quadrature hybrid coupler 508 is mounted to a second surface of the third substrate 502-3 that is opposite the first surface. Accordingly, the patch antenna 212, the third substrate 502-3, and the surface-mount quadrature hybrid coupler 508 are vertically stacked together. Similar to FIG. 5-1, the feeding vias 506-1 and 506-2 connect the ports 310-2 and 310-3 of the surface-mount quadrature hybrid coupler 508 to the feed pins 312-1 and 312-2 of the patch antenna 212 through the third substrate 502-3. Although not shown, other components of the radar system 104 (e.g., the transmitter 302, the receiver 304, or the system processor 218) can be disposed on the second surface of the third substrate 502-3 along with the surface-mount quadrature hybrid coupler 508. This can further reduce an overall size of the radar system 104.

In FIGS. 5-1 and 5-2, the compact circularly-polarized antenna 106 is shown to have a square or rectangular shape. Alternatively, the patch antenna 212, the substrates 502-1 to 502-3, and the ground plane 504 can be circular in shape to cause the compact circularly-polarized antenna 106 to have a circular shape. With a circular shape, the compact circularly-polarized antenna 106 can be readily integrated within smart devices 102 that have circular housings.

In some implementations, a size of the compact circularly-polarized antenna 106 can be further reduced for high-frequency based applications, such as micro-gesture detection. This reduced size enables the compact circularly-polarized antenna 106 to be implemented within other space-constrained smart devices 102, such as the smartphone 102-4 of FIG. 2. Additionally or alternatively, the compact circularly-polarized antenna 106 can be designed to transmit and receive wide-bandwidth signals, such as frequency-modulated (e.g., chirp) signals used for objection detection or gesture recognition. Generally, a bandwidth of the compact circularly-polarized antenna 106 is based on a substrate's permittivity or thickness. Therefore adjusting either of these characteristics in the substrates 502-1 to 502-3 of FIGS. 5-1 and 5-2 adjusts the bandwidth of the compact circularly-polarized antenna 106. Other feed configuration types, such as an aperture-coupled feed configuration can also be used instead of the pin-feed configuration to increase a bandwidth of the compact circularly-polarized antenna 106. As another example, multiple patch antennas 212 of different sizes and tuned to different frequencies can be vertically stacked together to increase the bandwidth of the compact circularly-polarized antenna 106. In other words, the multiple patch antennas 212 can be mostly overlapping relative to the substantially perpendicular axis between the multiple patch antennas 212 and the quadrature hybrid coupler 214.

By customizing a design of the compact circularly-polarized antenna 106 using any of the techniques described above, the compact circularly-polarized antenna 106 can transmit and receive signals with high frequencies (e.g., frequencies above 24 GHz) or large bandwidths (e.g., bandwidths between approximately 1 GHz and 4 GHz). With these frequencies and bandwidths, the radar system 104 can use the compact circularly-polarized antenna 106 to detect and recognize fine micro-gestures made by the user. Example types of micro-gestures include a knob-turning gesture whereby the user curls their fingers to grip an imaginary door knob and rotates their wrist, a spindle-twisting gesture whereby the user rubs their thumb and at least one other finger together, or a tapping gesture whereby the user taps their thumb and forefinger together.

Example Methods

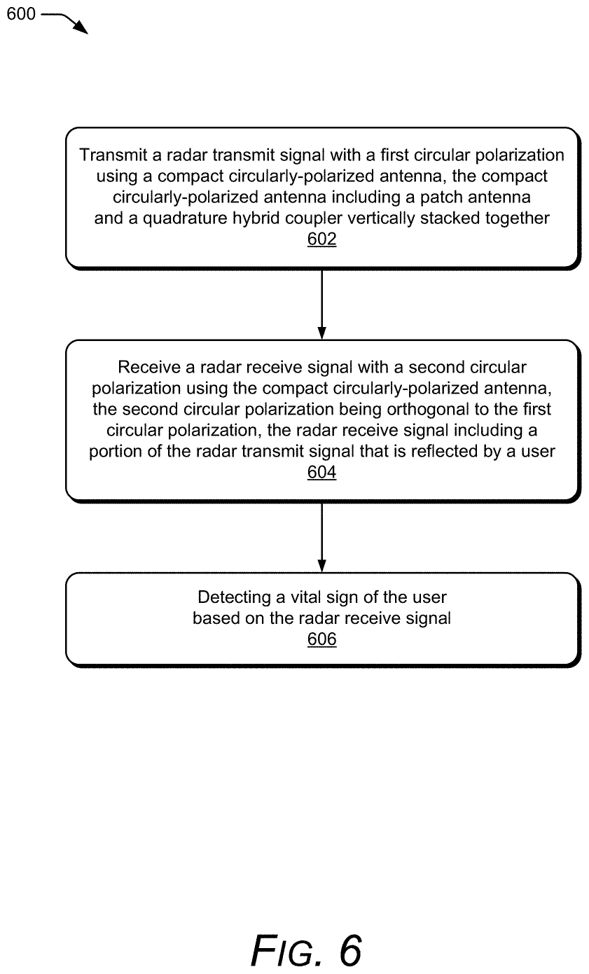

FIG. 6 depicts an example method 600 for performing operations of a smart device-based radar system capable of detecting human vital signs using a compact circularly-polarized antenna. Method 600 is shown as sets of operations (or acts) performed but not necessarily limited to the order or combinations in which the operations are shown herein. Further, any of one or more of the operations may be repeated, combined, reorganized, or linked to provide a wide array of additional and/or alternate methods. In portions of the following discussion, reference may be made to environment 100, FIG. 1, and entities detailed in FIGS. 2 through 5-2, reference to which is made for example only. The techniques are not limited to performance by one entity or multiple entities operating on one device.

At 602, a radar transmit signal is transmitted with a first circular polarization using a compact circularly-polarized antenna. The compact circularly-polarized antenna includes a patch antenna and a quadrature hybrid coupler, which are vertically stacked together. For example, the radar system 104 transmits the radar transmit signal 314 with the first circular polarization 316-1 using the compact circularly-polarized antenna 106, as shown in FIG. 3. The compact circularly-polarized antenna 106 includes the patch antenna 212 and the quadrature hybrid coupler 214, which are vertically stacked together as shown in FIGS. 5-1 and 5-2. The quadrature hybrid coupler 214 can be implemented using microstrip transmission lines, as shown in FIG. 5-1, or a surface-mount device, as shown in FIG. 5-2. In some cases, the quadrature hybrid coupler 214 is a ring-shaped quadrature hybrid coupler 400, as shown in FIG. 4. The vertical structure of the compact circularly-polarized antenna 106 reduces an overall lateral dimension of the compact circularly-polarized antenna 106 relative to other antenna designs and feed configuration types. In this manner, the radar system 104 can be implemented within a variety of different space-constrained smart devices 102.

At 604, a radar receive signal is received with a second circular polarization using the compact circularly-polarized antenna. The second circular polarization is orthogonal to the first circular polarization and the radar receive signal includes a portion of the radar transmit signal that is reflected by a user. For example, the radar system 104 receives the radar receive signal 318 with the second circular polarization 316-2 using the compact circularly-polarized antenna 106. The first circular polarization 316-1 and the second circular polarization 316-2 are orthogonal to each other. The first circular polarization 316-1 can be a RHCP and the second circular polarization 316-2 can be a LHCP, for instance. The radar receive signal 318 is a portion of the radar transmit signal 314 that is reflected by the user, as shown in FIG. 3.

At 606, a vital sign of the user is detected based on the radar receive signal. For example, the radar system 104 can detect a heartbeat and/or a respiration rate of the user based on the radar receive signal 318.

Example Computing System

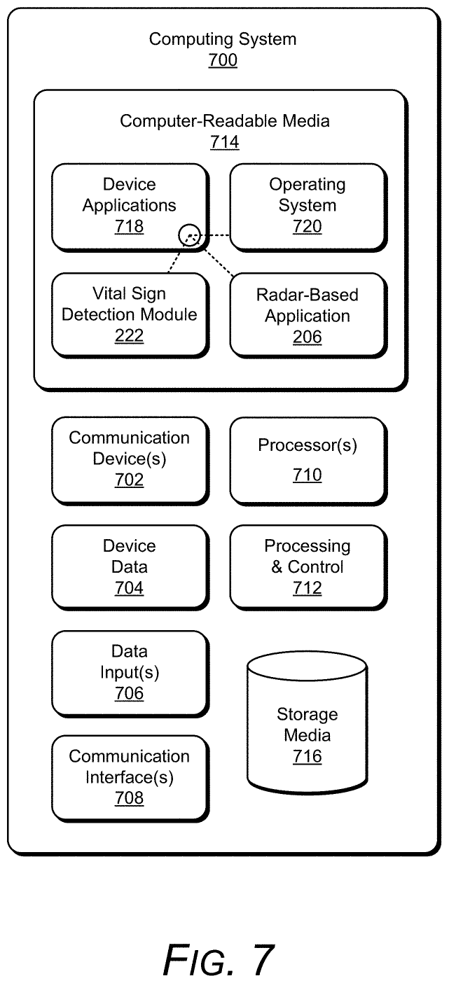

FIG. 7 illustrates various components of an example computing system 700 that can be implemented as any type of client, server, and/or computing device as described with reference to the previous FIG. 2 to implement human vital-sign detection.

The computing system 700 includes communication devices 702 that enable wired and/or wireless communication of device data 704 (e.g., received data, data that is being received, data scheduled for broadcast, or data packets of the data). The device data 704 or other device content can include configuration settings of the device, media content stored on the device, and/or information associated with a user of the device. Media content stored on the computing system 700 can include any type of audio, video, and/or image data. The computing system 700 includes one or more data inputs 706 via which any type of data, media content, and/or inputs can be received. In this case, the computing system 700 includes the compact circularly-polarized antenna 106, which receives the radar receive signal 318 as one of the data inputs 706. Other types of data inputs 706 include human utterances, user-selectable inputs (explicit or implicit), messages, music, television media content, recorded video content, and any other type of audio, video, and/or image data received from any content and/or data source.

The computing system 700 also includes communication interfaces 708, which can be implemented as any one or more of a serial and/or parallel interface, a wireless interface, any type of network interface, a modem, and as any other type of communication interface. The communication interfaces 708 provide a connection and/or communication links between the computing system 700 and a communication network by which other electronic, computing, and communication devices communicate data with the computing system 700.

The computing system 700 includes one or more processors 710 (e.g., any of microprocessors, controllers, and the like), which process various computer-executable instructions to control the operation of the computing system 700 and to enable techniques for, or in which can be embodied, human vital-sign detection. Alternatively or in addition, the computing system 700 can be implemented with any one or combination of hardware, firmware, or fixed logic circuitry that is implemented in connection with processing and control circuits which are generally identified at 712. Although not shown, the computing system 700 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

The computing system 700 also includes a computer-readable media 714, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. The disk storage device may be implemented as any type of magnetic or optical storage device, such as a hard disk drive, a recordable and/or rewritable compact disc (CD), any type of a digital versatile disc (DVD), and the like. The computing system 700 can also include a mass storage media device (storage media) 716.

The computer-readable media 714 provides data storage mechanisms to store the device data 704, as well as various device applications 718 and any other types of information and/or data related to operational aspects of the computing system 700. For example, an operating system 720 can be maintained as a computer application with the computer-readable media 714 and executed on the processors 710. The device applications 718 may include a device manager, such as any form of a control application, software application, signal-processing and control module, code that is native to a particular device, a hardware abstraction layer for a particular device, and so on.

The device applications 718 also include any system components, engines, or managers to implement human vital-sign detection. In this example, the device applications 718 include the vital-sign detection module 222 and the radar-based application 206.

Conclusion

Although techniques using, and apparatuses including a smart device-based radar system detecting human vital signs using a compact circularly-polarized antenna have been described in language specific to features and/or methods, it is to be understood that the subject of the appended claims is not necessarily limited to the specific features or methods described. Rather, the specific features and methods are disclosed as example implementations of smart device-based radar system detecting human vital signs using a compact circularly-polarized antenna.

1. A smart device comprising:
a radar system, the radar system including:
at least one compact circularly-polarized antenna, having dimensions that fit inside an exterior housing of the smart device, the at least one compact circularly-polarized antenna including:
a patch antenna positioned on a first plane and having a first feed pin and a second feed pin; and
a quadrature hybrid coupler positioned on a second plane, at least a portion of the quadrature hybrid coupler on the second plane overlapping a portion of the patch antenna on the first plane along an axis that is perpendicular to the first and second planes, the quadrature hybrid coupler including a first port, a second port coupled to the first feed pin of the patch antenna, a third port coupled to the second feed pin of the patch antenna, and a fourth port;
a transmitter coupled to the first port of the quadrature hybrid coupler and configured to transmit, via the at least one compact circularly polarized antenna, a radar transmit signal with a first circular polarization; and
a receiver coupled to the fourth port of the quadrature hybrid coupler and configured to receive, via the at least one compact circularly-polarized antenna, a radar receive signal with a second circular polarization that is orthogonal to the first circular polarization, the radar receive signal including a portion of the radar transmit signal that is reflected by a user; and
a human vital-sign detection module configured to detect a vital sign of the user based on the radar receive signal.

2. (canceled)

3. (canceled)

4. The smart device of claim 1, wherein:
the quadrature hybrid coupler includes a ring-shaped coupler;
the first port and the fourth port are positioned outside a circumference of the ring shaped coupler; and
the second port and the third port are positioned inside the circumference of the ring shaped coupler.

5. The smart device of claim 4, wherein:
the quadrature hybrid coupler includes four feed lines respectively coupled between the ring-shaped coupler and the first port, the second port, the third port, and the fourth port; and
the four feed lines are oriented along different radial axes of the ring-shaped coupler.

6. The smart device of claim 1, wherein the quadrature hybrid coupler comprises a surface-mount quadrature hybrid coupler.

7. The smart device of claim 1, wherein the transmitter is configured to transmit the radar transmit signal with a frequency between approximately two gigahertz (GHz) and six GHz.

8. (canceled)

9. (canceled)

10. A compact circularly-polarized antenna comprising:
a first substrate;
a second substrate;
a ground plane disposed between the first substrate and the second substrate;
a patch antenna disposed on the first substrate;
a quadrature hybrid coupler disposed on the second substrate; and
two feeding vias configured to respectively connect two ports of the quadrature hybrid coupler to two feed pins of the patch antenna through the first substrate, the ground plane, and the second substrate.

11-13. (canceled)

14. The compact circularly-polarized antenna of claim 10, wherein the quadrature hybrid coupler comprises a ring-shaped quadrature hybrid coupler having a curved microstrip transmission line formed in a circular shape.

15. The compact circularly-polarized antenna of claim 10, wherein the patch antenna, the first substrate, the ground plane, and the second substrate have circular shapes.

16. A method comprising:
transmitting a radar transmit signal with a first circular polarization using a compact circularly-polarized antenna, the compact circularly-polarized antenna including a patch antenna and a quadrature hybrid coupler vertically stacked together, the radar transmit signal comprising a continuous-sinusoidal signal having a relatively constant frequency;
receiving a radar receive signal with a second circular polarization using the compact circularly-polarized antenna, the second circular polarization being orthogonal to the first circular polarization, the radar receive signal including a portion of the radar transmit signal that is reflected by a user; and
detecting a vital sign of the user based on the radar receive signal.

17. The method of claim 16, wherein the detecting of the vital sign comprises detecting a heartbeat and a respiration rate of the user based on the radar receive signal.

18. The method of claim 17, wherein:
the radar receive signal comprises a frequency-modulated signal having a frequency that varies according to the heartbeat and the respiration rate of the user; and
the detecting of the vital sign comprises identifying frequencies of the radar receive signal that correspond to the heartbeat and the respiration rate.

19. The method of claim 17, further comprising:
detecting an abnormality associated with the user's heartbeat or respiration rate; and
alerting the user to the detected abnormality.

20. (canceled)

21. The smart device of claim 1, wherein the smart device comprises a smartphone.

22. The smart device of claim 1, wherein the smart device comprises:
a baby monitor;
a vehicle;
a wearable device;
a smart speaker;
a security camera;
a smart thermostat;
a gaming system; or
a home appliance.

23. The smart device of claim 1, wherein the at least one compact circularly-polarized antenna has lateral dimensions of approximately 50 millimeters by 50 millimeters.

24. The smart device of claim 1, wherein the at least one compact circularly-polarized antenna has a thickness of approximately 3 millimeters.

25. The compact circularly-polarized antenna of claim 10, wherein the first substrate and the second substrate comprise FR4 material.

26. The compact circularly-polarized antenna of claim 10, wherein:
the first substrate has a thickness of approximately 2 millimeters; and
the second substrate has a thicknesses of approximately 1 millimeter.

27. The compact circularly-polarized antenna of claim 10, wherein:
lateral dimensions of the patch antenna are approximately 28 millimeters by 28 millimeters; and
lateral dimensions of the first substrate, the ground, and the second substrate are approximately 50 millimeters by 50 millimeters.

28. The method of claim 16, wherein:
the transmitting of the radar transmit signal comprises transmitting the radar transmit signal using a transmit power of approximately zero decibel-milliwatts;
the relatively constant frequency is approximately 2.4 gigahertz; and
the detecting the vital sign of the user comprises detecting the vital sign of the user as a distance between the compact circularly-polarized antenna and the user is between approximately 15 centimeters to 60 centimeters.
